(19) United States
(12) Patent Application Publication
(10) Pub. No.: US 2025/0044298 A1
(43) Pub. Date: Feb. 6, 2025

(54) Oligosaccharide-Fluorescent Marker, and Preparation Method and Use thereof

(71) Applicant: JIANGNAN UNIVERSITY, Wuxi (CN)

(72) Inventors: Shuqin XU, Wuxi (CN); Jinghua CHEN, Wuxi (CN); Zhuqun WANG, Wuxi (CN); Xiaoli TANG, Wuxi (CN)

(21) Appl. No.: 18/920,143

(22) Filed: Oct. 18, 2024

Related U.S. Application Data
(63) Continuation of application No. PCT/CN2022/129774, filed on Nov. 4, 2022.

(30) Foreign Application Priority Data
Apr. 18, 2022 (CN) 202210403949.0

(51) Int. Cl. G01N 33/58; C07H 1/00; C07H 15/26; G01N 21/64

(52) U.S. Cl. CPC G01N 33/582; C07H 1/00; C07H 15/26; G01N 21/6428; G01N 21/6458; G01N 2021/6439

(57) ABSTRACT

The invention provides an oligosaccharide-fluorescent marker, and a preparation method and use thereof. The method includes reacting an oligosaccharide with a fluorescent agent in a solvent in the presence of a reducing agent to obtain the oligosaccharide-fluorescent marker. According to the oligosaccharide-fluorescent marker of the invention, an amino group in the fluorescent agent is subjected to a condensation reaction with a hemiacetal group at a reducing end of the oligosaccharide, to form an imine or Schiff base, and then the imine or Schiff base is reduced into a relatively stable secondary amine by using the reducing agent. In this way, the end of the oligosaccharide is fluorescently marked. The fluorescent group marked at the end has small influence on the biological activity of the oligosaccharide, and the marker has higher fluorescence intensity and is stable under physiological conditions.

### CROSS-REFERENCE TO RELATED APPLICATIONS

This application is a continuation of International Patent Application No. PCT/CN2022/129774, filed on Nov. 4, 2022, which claims the benefit of priority from Chinese Patent Application No. 202210403949.0, filed on Apr. 18, 2022. The content of the aforementioned applications, including any intervening amendments thereto, are incorporated herein by reference.

### FIELD OF THE INVENTION

The present invention relates to the field of biological technologies, and particularly to an oligosaccharide-fluorescent marker, and a preparation method and use thereof.

### DESCRIPTION OF THE RELATED ART

Chondroitin sulfate (CS) is a glycosaminoglycan, which participates in many biological functions such as cartilage regeneration, inflammatory reaction, development of the nervous system, and damage repair, and also in various vital activities, such as the growth, differentiation, and aging of cells, immune response and canceration, etc. It is closely related to human health and diseases. Therefore, it is of great significance to study the molecular structure and mechanism of action of oligosaccharides. In recent years, great progress has been made in the study of an oligosaccharide as an active ingredient or biomaterial. However, due to the seriously insufficient study on the pharmacokinetics of oligosaccharides, the further development and use of oligosaccharides as an active drug and biomaterial are limited. Up to now, fluorescent labeling of oligosaccharides has become one of the most promising methods for tracking oligosaccharides in vivo, aiming at detecting and identifying oligosaccharides, monitoring their metabolism in the body, tracking their distribution in cells and studying the biological activities of endogenous and synthetic glycolipids.

Fluorescent labeling of oligosaccharides shows the advantages of high sensitivity, visualization, and high intensity of detected signals, is used to study the metabolism and localization of labeled molecules in organisms and the interaction with cells, peptides and proteins, and is expected to become an effective way to study the principle and mechanism of action of saccharides. At present, this technology is widely used in the analysis and detection of nucleic acids and proteins, and has become a highly sensitive tool for the analysis and localization of biological macromolecules in biological research.

Currently, the commonly used derivatizing reagents for oligosaccharide-fluorescent markers include 2-aminoacridone (AMAC), and 2-aminopyridine (2-PA), etc. However, due to the limitation by quantum yield and fluorescence intensity, these derivatizing labeling reagents are only used for the separation and analysis of saccharides in chromatography, and cannot be used in research by microscopic fluorescence imaging.

### SUMMARY OF THE INVENTION

Therefore, the technical problem to be solved in the present invention is to overcome the problem existing in the prior art that oligosaccharides lack a fluorescent group and cannot be traced in vivo and overcome the influence of the labeling process on the biological activity of oligosaccharides.

To solve the above technical problems, the present invention provides an oligosaccharide-fluorescent marker, and a preparation method and use thereof. According to the present invention, a synthesized fluorescent agent containing an amino group is used as a fluorescent reagent, which undergoes a reductive amination reaction by the amino group with an aldehyde group or ketone group at a reducing end of an oligosaccharide, so that the fluorescence labeling of the oligosaccharide is realized without affecting the activity of the oligosaccharide. A first object of the present invention is to provide a method for preparing an oligosaccharide-fluorescent marker, which includes the following steps: reacting an oligosaccharide with a fluorescent agent in a solvent in the presence of a reducing agent to obtain the oligosaccharide-fluorescent marker.

In one embodiment of the present invention, the oligosaccharide has a structure shown below:

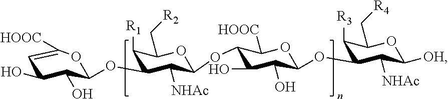

in which R₁, R₂, R₃, and R₄ are independently selected from —OH or —SO₃H, and n is an integer from 0 to 3.

In one embodiment of the present invention, the fluorescent agent is selected from the group consisting of 7-diethylamino-3-(4-aminophenyl)coumarin, phenanthroimidazole, 4-nitro-1,8-naphthalimide, 1-aminopyrene and any combination thereof.

7-Diethylamino-3-(4-aminophenyl)coumarin has a structure of

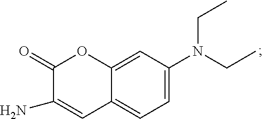 [SMILES: CCN(CC)c1ccc2cc(N)c(=O)oc2c1]

phenanthroimidazole has a structure of

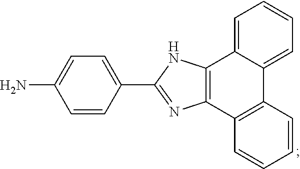 [SMILES: Nc1ccc(-c2nc3c4ccccc4c4ccccc4c3[nH]2)cc1]

4-nitro-1,8-naphthalimide has a structure of

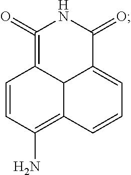

and 1-aminopyrene has a structure of

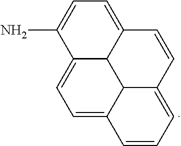 [SMILES: NC1=CC=C2C=CC3=CC=CC4=CC=C1C2C34]

In one embodiment of the present invention, the oligosaccharide-fluorescent marker has a structure shown below:

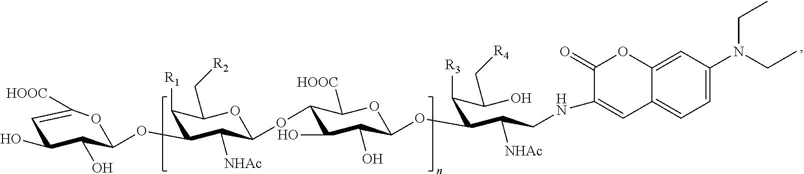

in which R₁, R₂, R₃, and R₄ are independently selected from —OH or —SO₃H, and n is an integer from 0 to 3.

In one embodiment of the present invention, the reducing agent is selected from the group consisting of 2-methylpyridine borane, sodium borohydride, sodium cyanoborohydride and any combination thereof.

In one embodiment of the present invention, the solvent is acetic and an organic solvent; and the organic solvent is selected from the group consisting of dimethyl sulfoxide, acetonitrile, N,N-dimethyl formamide and any combination thereof.

In one embodiment of the present invention, the reaction temperature is 40-80° C., and the reaction time is 2-10 hrs.

In one embodiment of the present invention, the molar ratio of the fluorescent agent, the oligosaccharide and the reducing agent is 0.5-4:1:0.5-4.

In one embodiment of the present invention, the method further includes a step of separating the oligosaccharide-fluorescent marker from the reaction solution. The step includes specifically: centrifugation, extraction with dichloromethane to obtain an aqueous phase, repeated water addition to and rotary evaporation of the aqueous phase, and then freeze-drying.

A second object of the present invention is to provide an oligosaccharide-fluorescent marker prepared by the method as described above.

In one embodiment of the present invention, the oligosaccharide-fluorescent marker can be prepared into a nano-material, which has no cytotoxicity when the concentration is 0.25-1.00 mg/mL when used in cell compatibility analysis. A second object of the present invention is to provide use of the oligosaccharide-fluorescent marker in the microscopic imaging and analysis of cells, where the concentration of the oligosaccharide-fluorescent marker is 1-50 μM during use.

Compared with the prior art, the technical solution of the present invention has the following advantages:

(1) According to the oligosaccharide-fluorescent marker of the present invention, an amino group in the fluorescent agent is subjected to a condensation reaction with a hemiacetal group at a reducing end of the oligosaccharide, to form an imine or Schiff base, and then the imine or Schiff base is reduced into a relatively stable secondary amine by using the reducing agent. In this way, the end of the oligosaccharide is fluorescently marked. The fluorescent group marked at the end has small influence on the biological activity of the oligosaccharide, and the marker has higher fluorescence intensity and is stable under physiological conditions.

(2) The oligosaccharide-fluorescent marker provided in the present invention is basically non-toxic to cells. Since the mechanism of action of chondroitin sulfate in different sulfation modes on nerve cells is not clear, the oligosaccharide is fluorescently labeled, to detect, track and visualize the oligosaccharide in oligodendrocyte precursor cells and astrocytes. Moreover, the oligosaccharide-fluorescent marker can be selectively taken by oligodendrocyte precursor cells, so that the oligosaccharide-fluorescent marker is suitable for use in imaging and analysis under a microscope.

### BRIEF DESCRIPTION OF THE DRAWINGS

To make the disclosure of the present invention more comprehensible, the present invention will be further described in detail by way of specific embodiments of the present invention with reference the accompanying drawings, in which:

FIG. 1 shows a synthesis route of an oligosaccharide-fluorescent marker in Example 1 of the present invention.

FIG. 2 shows a fluorescence spectrum of the oligosaccharide-fluorescent marker in Example 1 of the present invention.

FIG. 3 shows an IR and a UV spectrum of chondroitin 4mer, a fluorescent agent, and a 4mer-fluorescent marker in Example 1 of the present invention, in which a is an IR spectrum and b is an UV spectrum.

FIG. 4 shows the fluorescence performance analysis of the 4mer-fluorescent marker in Example 1 of the present invention, in which a shows the fluorescence intensity analysis and B shows the fluorescence stability analysis.

FIG. 5 shows the cytotoxicity analysis of the 4mer-fluorescent marker in Example 1 of the present invention to astrocytes.

FIG. 6 is a living cell image of the 4mer-fluorescent marker in Example 1 of the present invention in astrocytes.

FIG. 7 is a living cell image of the 4mer-fluorescent marker in Example 1 of the present invention in oligodendrocyte precursor cells.

FIG. 8 is a living cell image of the 4mer-fluorescent marker in Example 2 of the present invention in astrocytes.

FIG. 9 is a living cell image of a nano-material of the 4mer-fluorescent marker in Example 1 of the present invention when co-incubated with oligodendrocyte precursor cells and astrocytes.

### DETAILED DESCRIPTION OF THE PREFERRED EMBODIMENTS

The present invention will be further described below with reference to the accompanying drawings and specific examples, so that those skilled in the art can better understand and implement the present invention; however, the present invention is not limited thereto.

In the present invention, unless otherwise indicated, R₁, R₂, R₃, and R₄ are all-OH and n is 0 in the target compounds.

### Example 1

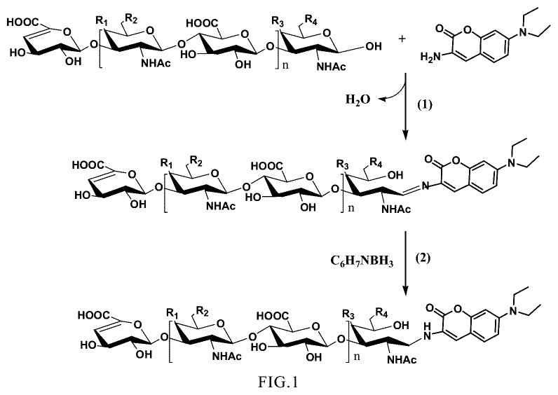

As shown in FIG. 1, an oligosaccharide-fluorescent marker was prepared through a method comprising specifically the following steps.

(1) Synthesis of 7-diethylamino-3-(4-aminophenyl)coumarin Fluorescent Agent

386 mg of 4-diethylamino-salicylic aldehyde, 293 mg of ethyl nitroacetate, 30 μL of piperidine, and 60 μL of glacial acetic acid were weighed and fully dissolved in 50 mL of n-butanol. The mixture was uniformly mixed, heated under reflux for 12 hrs in an oil bath at 100° C., and then cooled in an ice bath, to form an orange acicular material. After filtration under suction, the resulting solid was washed with 10 mL of n-butanol and 10 mL of petroleum ether, and then dried in a vacuum drying oven to obtain a primary product as an orange solid.

194 mg of anhydrous stannous chloride and 6 mL of concentrated hydrochloric acid were added to a 100 mL round-bottom flask. Then, 300 mg of the product in the previous step was slowly added to the 100 mL round-bottom flask and mixed well until they could react fully. Then, the mixed solution was reacted for 6 hrs with stirring at room temperature in the dark. After reaction, the reaction solution was adjusted to pH 13 with 1 M NaOH solution, and then extracted with three volumes of anhydrous diethyl ether. The upper organic layer was dried over anhydrous sodium sulfate, filtered under suction, and then dried in a vacuum drying oven at 40° C. to obtain the final product 7-diethylamino-3-(4-aminophenyl)coumarin as a red-brown solid that is a fluorescent agent.

### (2) Preparation of 4Mer-Fluorescent Marker

30 mg of chondroitin 4mer (prepared in laboratory) was added to 3 mL of 1 M 7-diethylamino-3-(4-aminophenyl)coumarin fluorescent agent (dissolved in DMSO:acetic acid=7:3), and reacted with shaking on a shaker for 30 min. Then equal volume of 1 M 2-methylpyridine borane dissolved in acetonitrile was added, and reacted for 6 hrs in a water bath at 60° C. in the dark. After the reaction, the reaction solution was centrifuged at 14 000 g for 20 min, and then extracted with dichloromethane. The organic phase was removed, and the aqueous phase was subjected to repeated water addition and rotary evaporation, and then freeze-dried to obtain the 4mer-fluorescent marker.

### Example 2

An oligosaccharide-fluorescent marker was prepared through a method comprising specifically the following steps.

### (1) Synthesis of Phenanthroimidazole Fluorescent Agent

583 mg of phenanthroquinone, 393 mg of p-nitrobenzaldehyde and 6 g of ammonium acetate were weighed and dissolved in 30 mL of glacial acetic acid. The mixture was mixed evenly, and heated for 12 hrs under reflux in an oil bath at 100° C. After filtration under suction, the resulting solid was washed with water and hexane, and then dried in a vacuum drying oven to obtain a primary product as an orange solid. 180 mg of anhydrous stannous chloride and 6 mL of concentrated hydrochloric acid were added to a 100 mL round-bottom flask. Then, 400 mg of the product in the previous step was slowly added to the 100 mL round-bottom flask and mixed well until they could react fully. Then, the mixed solution was reacted for 6 hrs with stirring at room temperature in the dark. After reaction, the reaction solution was adjusted to pH 13 with 1 M NaOH solution, and then extracted with three volumes of anhydrous diethyl ether. The upper organic layer was dried over anhydrous sodium sulfate, filtered under suction, and then dried in a vacuum drying oven at 40° C. to obtain the final product phenanthroimidazole as a red-brown solid that is a fluorescent agent.

### (2) Preparation of 4Mer-Fluorescent Marker

30 mg of chondroitin 4mer (prepared in laboratory) was added to 2 mL of 1 M phenanthroimidazole fluorescent agent (dissolved in DMF:acetic acid=7:3), and reacted with shaking on a shaker for 30 min. Then equal volume of 1 M sodium cyanoborohydride dissolved in acetonitrile was added, and reacted for 10 hrs in a water bath at 40° C. in the dark. After the reaction, the reaction solution was centrifuged at 14 000 g for 20 min, and then extracted with dichloromethane. The organic phase was removed, and the aqueous phase was subjected to repeated water addition and rotary evaporation, and then freeze-dried.

### Example 3

An oligosaccharide-fluorescent marker was prepared through a method comprising specifically the following steps.

(1) Synthesis of 4-nitro-1,8-naphthalimide Fluorescent Agent

770 mg of acenaphthene was mixed with 3 mL of acetic acid, added with 4 mL of nitric acid, and reacted at 80° C. for 1 hr. After cooling, a yellow crystal was precipitated out. Na₂Cr₂O₇·2H₂O was added and reacted at 100° C. for 3 hrs. After filtration under suction, the resulting solid was washed with 30 mL of water, added with 30 mL of Na₂CO₃, and filtered. The filtrate was adjusted to pH 2 with hydrochloric acid, and filtered. The filter cake was washed with water to neutrality, and dried in a vacuum drying oven at 120° C., to obtain a preliminary solid as a pale yellow solid.

In a 250 mL three-neck flask, 500 mg of 4-nitronaphthalic anhydride was added. 10 ml of a solution containing 2.4 g of Na₂S·9H₂O and 1.3 g of NaHCO₃ was added dropwise to the reaction flask with stirring (over 15 min). Then the system was controlled to further react for 40 min at 80° C. After the reaction, the solution was boiled to remove the generated H₂S, and then filtered. The filtrate was adjusted to about pH 5 with hydrochloric acid, and cooled in an ice bath to precipitate out a pale yellow acicular, which was filtered and dried to obtain 4-nitro-1,8-naphthalimide that is a fluorescent agent.

### (2) Preparation of 4Mer-Fluorescent Marker

30 mg of chondroitin 4mer (prepared in laboratory) was added to 3 mL of 1 M 4-nitro-1,8-naphthalimide fluorescent agent (dissolved in DMSO:acetic acid=7:3), and reacted with shaking on a shaker for 30 min. Then equal volume of 1 M 2-methylpyridine borane dissolved in acetonitrile was added, and reacted for 2 hrs in a water bath at 80° C. in the dark. After the reaction, the reaction solution was centrifuged at 14 000 g for 20 min, and then extracted with dichloromethane. The organic phase was removed, and the aqueous phase was subjected to repeated water addition and rotary evaporation, and then freeze-dried.

### Example 4

An oligosaccharide-fluorescent marker was prepared through a method comprising specifically the following steps.

(1) Synthesis of 1-aminopyrene Fluorescent Agent

1 g of pyrene was dissolved in 10 mL of acetic acid, 11 mL of 68% concentrated nitric acid was slowly added dropwise at 20° C. over 1 hrs, and heated under reflux for 2 hrs. After the reaction, 10 mL of water was added into the reaction system to obtain a brownish red precipitate, which was recrystallized in acetic acid after filtration under suction and dried in a vacuum drying oven to obtain a brownish red powder.

800 mg of the reaction product from the previous step and 500 mg of Pd/C catalyst were added to 10 mL of ethanol, and slowly heated to reflux. 1 mL of hydrazine hydrate was added dropwise under reflux over 3 hrs. After the reaction was completed, the reaction solution was filtered while hot. The filtrate was cooled to room temperature, and filtered to obtain a yellow crystal, namely 1-aminopyrene that is a fluorescent agent.

### (2) Preparation of 4Mer-Fluorescent Marker

40 mg of chondroitin 4mer (prepared in laboratory) was added to 3 mL of 1 M 1-aminopyrene fluorescent agent (dissolved in DMF:acetic acid=7:3), and reacted with shaking on a shaker for 30 min. Then equal volume of 1 M sodium borohydride dissolved in acetonitrile was added, and reacted for 10 hrs in a water bath at 60° C. in the dark. After the reaction, the reaction solution was centrifuged at 14 000 g for 20 min, and then extracted with dichloromethane. The organic phase was removed, and the aqueous phase was subjected to repeated water addition and rotary evaporation, and then freeze-dried.

### Comparative Example 1

(1) Synthesis of 7-diethylamino-3-(4-aminophenyl)coumarin Fluorescent Agent

386 mg of 4-diethylamino-salicylic aldehyde, 293 mg of ethyl nitroacetate, 30 μL of piperidine, and 60 μL of glacial acetic acid were weighed and fully dissolved in 50 mL of n-butanol. The mixture was uniformly mixed, heated under reflux for 12 hrs in an oil bath at 100° C., and then cooled in an ice bath, to form an orange acicular material. After filtration under suction, the resulting solid was washed with 10 mL of n-butanol and 10 mL of petroleum ether, and then dried in a vacuum drying oven to obtain a primary product as an orange solid.

194 mg of anhydrous stannous chloride and 6 mL of concentrated hydrochloric acid were added to a 100 mL round-bottom flask. Then, 300 mg of the product in the previous step was slowly added to the 100 mL round-bottom flask and mixed well until they could react fully. Then, the mixed solution was reacted for 6 hrs with stirring at room temperature in the dark. After reaction, the reaction solution was adjusted to pH 13 with 1 M NaOH solution, and then extracted with three volumes of anhydrous diethyl ether. The upper organic layer was dried over anhydrous sodium sulfate, filtered under suction, and then dried in a vacuum drying oven at 40° C. to obtain the final product 7-diethylamino-3-(4-aminophenyl)coumarin as a red-brown solid that is a fluorescent agent.

### (2) Preparation of 4Mer-Fluorescent Marker

30 mg of chondroitin 4mer (prepared in laboratory) was added to 3 mL of 1 M 7-diethylamino-3-(4-aminophenyl)coumarin fluorescent agent (dissolved in DMSO), and reacted with shaking on a shaker for 30 min. Then equal volume of 1 M 2-methylpyridine borane dissolved in acetonitrile was added, and reacted for 6 hrs in a water bath at 60° C. in the dark. After the reaction, the reaction solution was centrifuged at 14 000 g for 20 min, and then extracted with dichloromethane. The organic phase was removed, and the aqueous phase was subjected to repeated water addition and rotary evaporation. Due to the absence of acetic acid, the obtained product does not fluoresce.

### Comparative Example 2

(1) Synthesis of 7-diethylamino-3-(4-aminophenyl)coumarin Fluorescent Agent

386 mg of 4-diethylamino-salicylic aldehyde, 293 mg of ethyl nitroacetate, 30 μL of piperidine, and 60 μL of glacial acetic acid were weighed and fully dissolved in 50 mL of n-butanol. The mixture was uniformly mixed, heated under reflux for 12 hrs in an oil bath at 100° C., and then cooled in an ice bath, to form an orange acicular material. After filtration under suction, the resulting solid was washed with 10 mL of n-butanol and 10 mL of petroleum ether, and then dried in a vacuum drying oven to obtain a primary product as an orange solid.

194 mg of anhydrous stannous chloride and 6 mL of concentrated hydrochloric acid were added to a 100 mL round-bottom flask. Then, 300 mg of the product in the previous step was slowly added to the 100 mL round-bottom flask and mixed well until they could react fully. Then, the mixed solution was reacted for 6 hrs with stirring at room temperature in the dark. After reaction, the reaction solution was adjusted to pH 13 with 1 M NaOH solution, and then extracted with three volumes of anhydrous diethyl ether. The upper organic layer was dried over anhydrous sodium sulfate, filtered under suction, and then dried in a vacuum drying oven at 40° C. to obtain the final product 7-diethylamino-3-(4-aminophenyl)coumarin as a red-brown solid that is a fluorescent agent.

### (2) Preparation of 4Mer-Fluorescent Marker

30 mg of chondroitin 4mer (prepared in laboratory) was added to 3 mL of 1 M 7-diethylamino-3-(4-aminophenyl)coumarin fluorescent agent (dissolved in DMSO:acetic acid=7:3), and reacted with shaking on a shaker for 30 min. Then equal volume of 1 M 2-methylpyridine borane dissolved in acetonitrile was added, and reacted for 6 hrs in a water bath at 60° C. in the dark. After the reaction, the reaction solution was centrifuged at 14 000 g for 20 min, and the product was purified by Sephadex G-25, and fluorescently adsorbed on the column, which shortened the life of the 4mer-fluorescent marker.

### Test Example 1

The fluorescent agent and 4mer-fluorescent marker prepared in Example 1 were analyzed by fluorescence spectrometry.

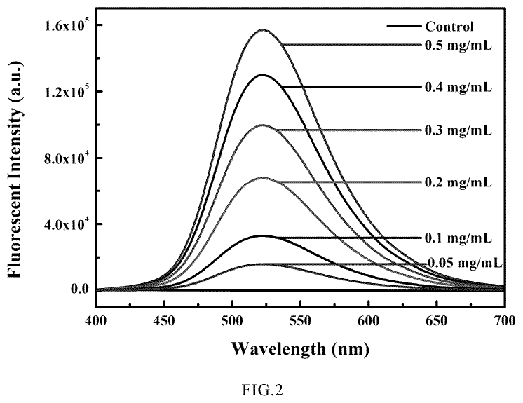

Fluorescent agent solutions of 0.25, 0.5, 1, 2, and 4 μg/mL and 4mer-fluorescent marker solutions of 0.05, 0.1, 0.2, 0.3, 0.4, and 0.5 mg/mL were prepared. The fluorescence intensity of the solutions were detected by a fluorescence spectrophotometer using an excitation wavelength of 380 nm, an emission wavelength of 400-650 nm, and a bandwidth of excitation and emission light of 5 nm. The results are shown in FIG. 2. The results show that the 4mer-fluorescent marker has an emission band centered at 522 nm, which is blue shifted to some extent compared with the fluorescent agent having an emission band centered at 490 nm. Because the fluorophore bound to the 4mer has changed the absorbance and emission of the fluorophore. According to the standard curve of the fluorescent agent and the fluorescence intensity of the marker, the average labeling rate of the 4mer-fluorescent marker is calculated to be 3.64%.

### Test Example 2

The chondroitin 4mer, 7-diethylamino-3-(4-aminophenyl)coumarin fluorescent agent (B), and 4mer-fluorescent marker (4mer-B) prepared in Example 1 were analyzed by IR and UV spectrometry.

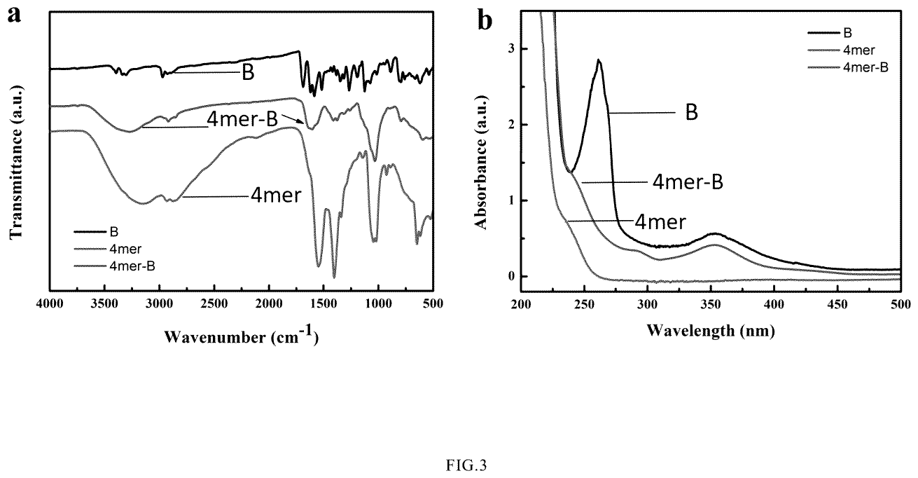

After freeze-drying under vacuum, the samples of chondroitin 4mer, 7-diethylamino-3-(4-aminophenyl)coumarin fluorescent agent (B), and 4mer-fluorescent marker (4mer-B) were respectively placed under the probe of an IR spectrometer, the probe was moved down to compact the sample, and the IR spectra of the samples were detected. As shown in FIG. 3a, the IR spectrum of 4mer-B shows an obvious characteristic absorption peak at 1616 cm-1, which can be attributed to the stretching vibration peak of C—N bond formed by a reductive amination reaction of the reductive terminal of the chondroitin 4mer with the amino group on the fluorescent agent, indicating that the chondroitin 4mer is successfully modified with the fluorescent agent.

100 μg/mL chondroitin 4mer, fluorescent agent, and 4mer-fluorescent marker were respectively placed in a cuvette in a UV spectrometer, and the ultraviolet-visible spectra in the range of 800-200 nm were detected. The results as shown in FIG. 3b. The chondroitin 4mer and the 4mer-fluorescent marker both have absorption at 232 nm, and the 4mer-fluorescent marker has an absorption peak at 350 nm, which is the same as that of the fluorescent agent in an aqueous solution. Therefore, the UV-Vis results further prove that the chondroitin 4mer is successfully modified with the fluorescent agent.

### Test Example 3

The influence of pH value on the fluorescence intensity and stability of the 4mer-fluorescent marker (4mer-B) prepared in Example 1 was analyzed.

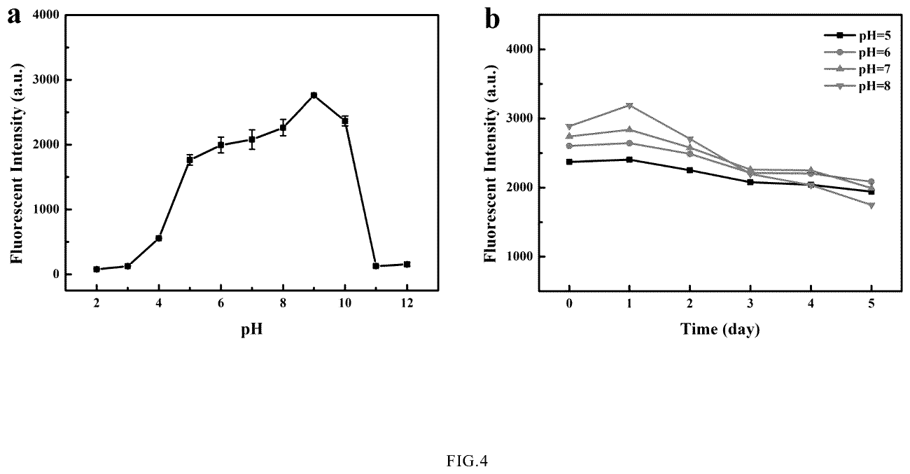

The 4mer-fluorescent marker was dissolved in a buffer (pH 2-12), to give a final concentration of 10 μg/mL. The fluorescence intensity of the 4mer-fluorescent marker at various pH values was measured by a fluorescence spectrophotometer, and the influence of pH value on fluorescence intensity was investigated. The measurement was carried out under conditions including an excitation wavelength of 380 nm, an emission wavelength of 522 nm, and a bandwidth of excitation light and emission light of 5 nm. The samples were stored in the dark for 5 days at room temperature, and the fluorescence intensity of each sample was measured every 24 hrs. The changes of the fluorescence intensity of the samples at various pH values as a function of storage time were observed. As shown in FIG. 4a, the results showed that the 4mer-fluorescent marker is sensitive to pH and has high fluorescence intensity under physiological conditions. The fluorescence stability is shown in FIG. 4b, showing that the fluorescence intensity decreases slightly after being stored at room temperature for 5 days, but the amplitude is limited.

### Test Example 4

Cytocompatibility analysis was carried out on the 4mer-fluorescent marker prepared in Example 1.

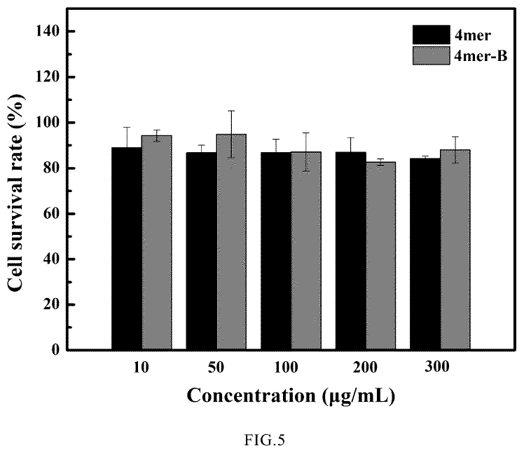

The chondroitin 4mer-fluorescent marker was co-incubated with astrocytes, and the cell survival rate was detected by MTT method. The results are shown in FIG. 5. The results show that when the 4mer and the 4mer-fluorescent marker have a concentration in the range of 0-300 μg/mL, the cell viability of astrocytes is above 80%, indicating that the 4mer and the 4mer-fluorescent marker are less toxic to astrocytes and will not affect the experimental results, so they can be used to study the cell uptake.

### Test Example 5

### (1) Analysis of 4Mer-Fluorescent Marker Prepared in Example 1 by Laser Confocal Imaging:

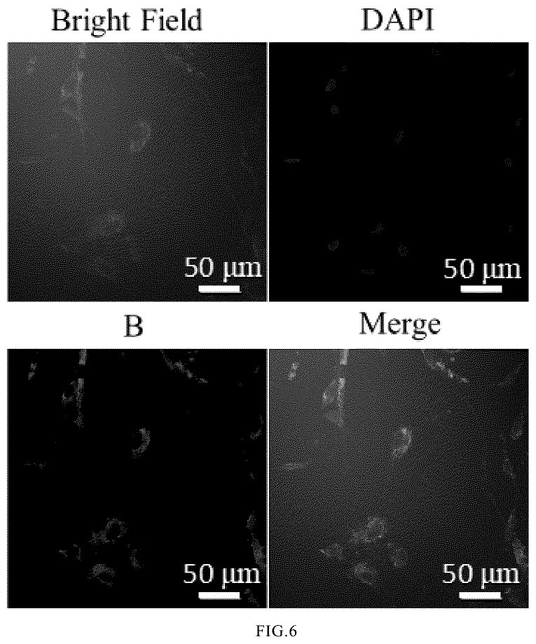

Astrocytes in good growth state were digested and dispersed with an appropriate amount of trypsin for 5 min to prepare a cell suspension. The cells were plated on a confocal dish coated with PLL overnight at a density of 5×10⁴ cells/mL, and incubated for 24 hrs in an incubator at 37° C. and 5% CO₂. The upper medium was removed after the cell adhesion. 100 μg/mL 4mer-7-diethylamino-3-(4-aminophenyl)coumarin fluorescent marker was added. After the cells were incubated for 24 hrs, the original culture medium was aspirated off, and 4% paraformaldehyde fix solution was added, with which the cells were fixed for 30 min. The cells were washed three times with PBS. The cells incubated with 100 μg/mL 4mer-fluorescent marker was stained with an appropriate amount of DAPI for 15-20 min, washed three times with PBS, and photographed under a laser confocal microscope for data acquisition. The results are shown in FIG. 6, indicating that the 4mer-fluorescent marker emits strong green fluorescence and can be internalized by astrocytes effectively.

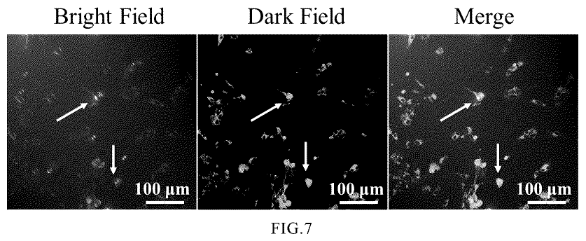

Oligodendrocyte precursor cells in good growth state were digested and dispersed with an appropriate amount of trypsin for 5 min to prepare a cell suspension. The cells were plated on a confocal dish coated with PLL overnight at a density of 5×10⁴ cells/mL, and incubated for 24 hrs in an incubator at 37° C. and 5% CO₂. The upper medium was removed after the cell adhesion. 100 g/mL 4mer-fluorescent marker was added. After the cells were incubated for 24 hrs, the original culture medium was aspirated off, and 4% paraformaldehyde fix solution was added, with which the cells were fixed for 30 min. The cells were washed three times with PBS. The cells incubated with 100 μg/mL 4mer-fluorescent marker was stained with an appropriate amount of DAPI for 15-20 min, washed three times with PBS, and photographed under a laser confocal microscope for data acquisition. The results are shown in FIG. 7. Due to the presence of abundant saccharide binding protein receptors on the surface of oligodendrocyte precursor cells, the ability to bind and the uptake of the labeled chondroitin 4mer by the cells are further enhanced.

### (2) Analysis of 4Mer-Fluorescent Marker Prepared in Example 2 by Laser Confocal Imaging:

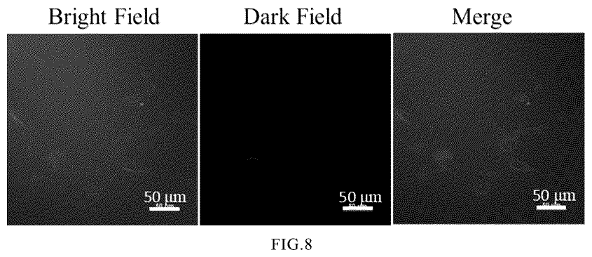

Astrocytes in good growth state were digested and dispersed with an appropriate amount of trypsin for 5 min to prepare a cell suspension. The cells were plated on a confocal dish coated with PLL overnight at a density of 5×10⁴ cells/mL, and incubated for 24 hrs in an incubator at 37° C. and 5% CO₂. The upper medium was removed after the cell adhesion. 100 μg/mL 4mer-phenanthroimidazole fluorescent marker was added. After the cells were incubated for 24 hrs, the original culture medium was aspirated off, and 4% paraformaldehyde fix solution was added, with which the cells were fixed for 30 min. The cells were washed three times with PBS. The cells incubated with 100 μg/mL 4mer-fluorescent marker was stained with an appropriate amount of DAPI for 15-20 min, washed three times with PBS, and photographed under a laser confocal microscope for data acquisition. The results are shown in FIG. 8, indicating that the 4mer-fluorescent marker emits strong blue fluorescence and can be internalized by astrocytes effectively.

### Test Example 6

8 mg of the 4mer-fluorescent marker in Example 1, 42 mg of EDC and 60 mg of NHS were dissolved in 5 mL of Tris, adjusted to pH 5.5, and activated for 50 min. The solution was adjusted to pH 7.4, and a suspension of aminated cellulose nanowhiskers (20 mg/mL, 1 mL) was added, and stirred for 24 hrs in the dark. The aminated cellulose nanowhiskers was washed by centrifugation at 10000 r/min for 10 min, followed by dialysis and freeze-drying to obtain the product functionalized cellulose nanowhiskers (N4B).

### (2) Cytocompatibility Analysis of 4Mer-Fluorescent Marker Nanoparticles

A 96-well plate was coated with 0.05 mg/mL PLL for 8 hrs and washed with PBS for 3 times. A mixed cell suspension of oligodendrocyte precursor cells and astrocytes was inoculated into the 96-well plate at a density of 100,000 cells/mL. A mixed cell culture medium containing 0.1 mg/mL, 0.25 mg/mL, 0.50 mg/mL, 0.75 mg/mL or 1.00 mg/mL N4B was prepared. 100 μL of corresponding mixed cell culture solution was added to each well. The culture medium was removed after 1- and 3-day incubation. 100 μL of MTT was added to each well, and the cells were incubated for 4 hrs in a constant-temperature incubator at 37° C. MTT was removed, 100 μL of DMSO was added, and the cells were incubated for 30 min on a shaker at 37° C. The absorbance at 570 nm was detected on a microplate reader. The results show that N4B in the concentration range of 0.25-1.00 mg/mL has no cytotoxicity.

### (3) Analysis of 4Mer-Fluorescent Marker Nanoparticles by Laser Confocal Imaging

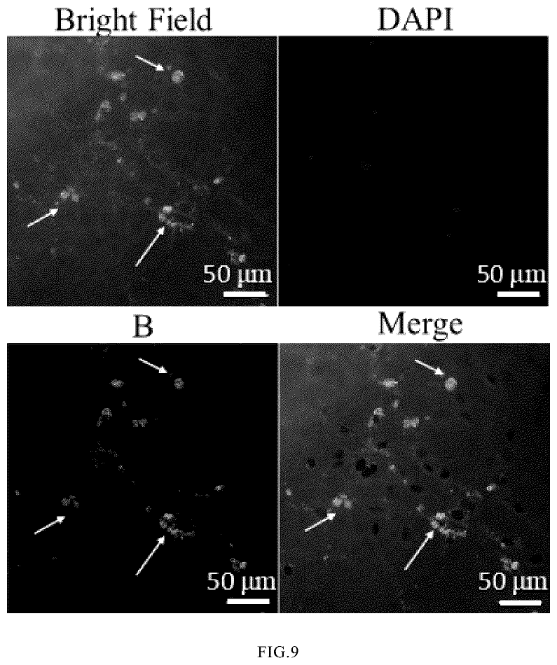

A confocal dish was coated with 0.05 mg/mL PLL. After 8 hrs, the PLL solution was aspirated off and the confocal dish was washed with PBS. A mixed cell suspension of oligodendrocyte precursor cells and astrocytes were inoculated on a confocal dish at a density of 100,000 cells/mL. A mixed cell culture medium containing 1.00 mg/mL N4B was prepared. The cells were incubated in a constant-temperature incubator at 37° C. and 5% CO₂ for 24 hrs. The culture medium was aspirated off. The cells were fixed for 10 min with 4% paraformaldehyde fix solution, and washed with PBS. 100 μL of PBS was added to the confocal dish to be detected. As shown in FIG. 9, the results show that after 24-h incubation, the cells basically retain the normal morphology, indicating that N4B has biocompatibility. Under the same conditions, due to the presence of abundant saccharide binding protein receptors on the surface of oligodendrocyte precursor cells, the ability to bind the labeled chondroitin 4mer is enhanced, and the 4mer-fluorescent marker nanoparticles are selectively ingested by the oligodendrocyte precursor cells.

Apparently, the above-described embodiments are merely examples provided for clarity of description, and are not intended to limit the implementations of the present invention. Other variations or changes can be made by those skilled in the art based on the above description. The embodiments are not exhaustive herein. Obvious variations or changes derived therefrom also fall within the protection scope of the present invention.

What is claimed is:

1. A method for preparing an oligosaccharide-fluorescent marker, comprising a step of: reacting an oligosaccharide with a fluorescent agent in a solvent in the presence of a reducing agent to obtain the oligosaccharide-fluorescent marker.

2. The method for preparing an oligosaccharide-fluorescent marker according to claim 1, wherein the oligosaccharide has a structure shown below: 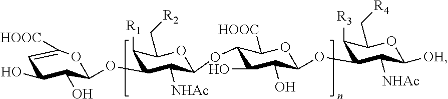
in which R₁, R₂, R₃, and R₄ are independently selected from —OH or —SO₃H, and n is an integer from 0 to 3.

3. The method for preparing an oligosaccharide-fluorescent marker according to claim 1, wherein the fluorescent agent is selected from the group consisting of 7-diethylamino-3-(4-aminophenyl)coumarin, phenanthroimidazole, 4-nitro-1,8-naphthalimide, 1-aminopyrene and any combination thereof.

4. The method for preparing an oligosaccharide-fluorescent marker according to claim 1, wherein the reducing agent is selected from the group consisting of 2-methylpyridine borane, sodium borohydride, sodium cyanoborohydride and any combination thereof.

5. The method for preparing an oligosaccharide-fluorescent marker according to claim 1, wherein the solvent is acetic acid and an organic solvent.

6. The method for preparing an oligosaccharide-fluorescent marker according to claim 5, wherein the organic solvent is selected from the group consisting of dimethyl sulfoxide, acetonitrile, N,N-dimethyl formamide and any combination thereof.

7. The method for preparing an oligosaccharide-fluorescent marker according to claim 1, wherein the reaction temperature is 40-80° C. and the reaction time is 2-10 hrs.

8. The method for preparing an oligosaccharide-fluorescent marker according to claim 1, wherein the molar ratio of the fluorescent agent, the oligosaccharide and the reducing agent is 0.5-4:1:0.5-4.

9. An oligosaccharide-fluorescent marker prepared by the method according to claim 1.

10. Use of the oligosaccharide-fluorescent marker according to claim 9 in the microscopic imaging and analysis of cells, wherein the concentration of the oligosaccharide-fluorescent marker is 1-50 μM during use.
